(19) United States
(12) Patent Application Publication
(10) Pub. No.: US 2006/0188394 A1
(43) Pub. Date: Aug. 24, 2006

(54) System comprising effectors and elastomers modified by variable-volume receptors, method for the production and use thereof

(76) Inventor: Hans-Jorg Schneider, Saarbrucken (DE)

Correspondence Address: Stephen D Scanlon;Jones Day, 901 Lakeside Avenue, Cleveland, OH, 44114, US

(21) Appl. No.: 10/562,607

(22) PCT Filed: Jun. 28, 2004
(86) PCT No.: PCT/EP04/06996

§ 371 Date: Dec. 28, 2005

(30) Foreign Application Priority Data
Jul. 4, 2003 (DE) 103 30 269.7

(51) Int. Cl. G01N 33/50

(52) U.S. Cl. 422/56

(57) ABSTRACT

System comprising at least one effector and one receptor-modified elastomer, characterized in that upon bringing into contact the at least one effector with the at least one receptor-modified elastomer, a change in volume is triggered, which preferably is reversible, by means of forming a selective non-covalent or covalent bond between receptors and the at least one effector in the receptor-modified elastomer.

The present invention relates to a system comprising at least one effector and at least one receptor modified elastomer, wherein, upon bringing into contact of the at least one effector with the at least one receptor modified elastomer, the elastomer is experiencing to a reversible or a non-reversible change in volume by means of forming a selective non-covalent or covalent bond between receptors and the at least one effector. A further object of the invention is a process for the manufacture of a system comprising the reaction of functionalized polymers with receptors or the reaction of functionalized monomers with receptors and subsequent polymerization, as well as the use of the system for flow-control, as an actuator as a sensor or as a sensor array, for setting free or taking up active ingredients or as sealing material. The invention also relates to an element for flow-control, an actuator for performing mechanical movements, a sensor for chemical, mechanical, electrical, electromechanical, magnetic or optical signals or a sensor array, a device for setting free or taking up active ingredients as well as a seal, which can be manufactured by making use of the system.

Elastomers that react with a change in volume upon changes in the chemical environment are already known.

Patent application WO 02/071994 discloses hydrogels that change their volume upon changes of the pH-value. These are manufactured by reacting ethylenically unsaturated monomers and polymers that carry groups that can be ionized with network forming components and polymerization catalysts. The hydrogels expand under basic conditions if the elastomer network contains carboxylic groups. Acrylic acid and methacrylic acid are used as ethylenically unsaturated monomers, also in conjunction with acryl amide or 2-hydroxy ethylmethacrylate. Expansion under acidic conditions is possible if the network contains amine groups. Therefore, in the first case expansion occurs upon deprotonation, whereas in the second case, expansion occurs upon protonation. Elastomers that enlarge their volume upon protonation as well as upon deprotonation are not disclosed in said document.

Hydrogels that change their volume by means of expansion upon deprotonation, i. e. under basic conditions, are known from U.S. Pat. No. 6,173,865. As can be seen from FIG. 3 of said patent, protonation, on the other hand, does not lead to an increase in volume. The hydrogels are manufactured by means of reacting monomers that can be polymerized and that contain sulfone groups, with acrylic acid amides in the presence of network forming compounds such as N,N′-methylen bisisocyanate.

U.S. Pat. No. 5,415,864 discloses networks of hydrogels that are manufactured from ethylenically unsaturated comonomers that contain no groups that can be ionized, ethylenically unsaturated comonomers with groups that can be ionized and a network forming compound that contains an aromatic azo-coupling. Groups that can be ionized are the carboxylic and the sulfanate group, groups that can not be ionized are, for example, amide, ester, phenyl and nitrile groups. The hydrogels expand upon increasing the pH-value by means of deprotonation. FIGS. 1, 3 and 4 of this patent show that no increase in volume can be achieved by means of protonation.

U.S. Pat. No. 5,226,902 also discloses hydrogels that change their volume upon changing of the pH-value. Hydrogels that expand upon increasing pH-value or, respectively, contract upon decreasing pH-value are manufactured by means of polymerization of monomers that carry carboxylic groups. In addition, hydrogels are known from this patent that contract upon increasing pH-value or expand upon decreasing pH-value. These are manufactured by means of polymerization of monomers with amine groups. Furthermore, it is known from this patent that the hydrogels can also alter their volume in the presence of glucose and glucose oxidase. Ultimately, however, this effect can be traced back to the fact that the pH-value of the environment of the hydrogel changes upon the glucose oxidase acting on the glucose. Therefore, this change in volume is only based on a change in the pH-value.

As can be deducted from these documents from the prior art, the use of said hydrogels is limited to biomedical applications and to changes that are sensitive to the pH-value. These products can be used, for example, in medical devices for setting free active ingredients, for example in implants, however, only as in consequence of changes in the pH-value.

Furthermore, networks of polymer compositions are also known experiencing a macroscopic change in volume in response to the effect of salt water. These polymer compositions consist of a mixture of a network of polymers containing acidic groups and a network of polymers containing basic groups. The acidic as well as the basic type of polymer already are swellable as individual components with water, wherein the change of volume of the acidic compound can be traced back to the loss of protons, whereas the change of volume of the basic compound can be traced back to accepting protons. Ionic bonds are formed between the acidic and the basic polymer particles upon mixing the two types of polymers. The change in volume is achieved by intercalating water between the particles, thereby effecting a widening of the network and effecting an enhancement of the take-up of water vis-à-vis the changes in volume of the individual components (U.S. Pat. No. 6,333,105).

One object according to the present invention can be seen to provide further systems, with which a change in volume can be effected selectively by means of the effect of certain substances in certain ranges of concentration.

This object can be solved by bringing into contact receptor modified elastomers with external effectors, wherein the change in volume is effected by the formation of selective non-covalent bonds or also covalent bonds between effectors and receptors of the elastomers.

One object according to the present invention, therefore, is a system comprising at least one effector and at least one receptor modified elastomer, characterized in that a change in volume is effected upon bringing into contact the at least one effector with the at least one receptor modified elastomer by means of forming a selective non-covalent or covalent bond between the receptors and the at least one effector of the receptor modified elastomer.

The change in volume may be reversible as well as non-reversible.

In a preferred embodiment, to change in volume is reversible.

In a preferred embodiment, the change in volume upon the formation of the selective non-covalent or covalent bond is an increase of volume. If this bond is cleaved again by means of giving away the effector to its environment, a decrease of volume occurs.

The system comprising at least one effector and a receptor modified elastomer has an advantage over the systems described in the prior art by the fact that a multitude of substances can be used as effectors, furthermore that selective interactions occur thereby and furthermore that several effectors operate simultaneously and cooperatively. In the meaning of the present invention, essential for the change in volume is only that a selective non-covalent or covalent bond is formed between effectors and receptors of the elastomer.

Therefore, systems in which the change in volume is not mainly effected by means of the formation of a non-covalent bond or a covalent bond between effectors and receptors, but by means of other mechanisms, are excluded from the present invention. For example, a change in volume may be caused by unfolding of the polymer chains contained in the elastomer. Such systems, for example, contain water that diffuses into the elastomer. The unfolding of polymer chains triggered thereby results in a swelling of the elastomer. The mechanism is described in WO 02/071994. This applies in an almost analogous manner also to systems in which the polymer chains of the elastomer are unfolded by means of diffusing of organic solvents under swelling of the elastomer.

The term elastomer according to the present invention is meant to comprise all polymers that are elastic or that at least comprise elastic parts. In a preferred embodiment, these are networks of polymers. Elasticity or partial elasticity, respectively, can be characterized according to the common physical methods known in the art, for example by means of determining the memory module.

Receptors are to be understood to comprise all compounds or derivatives of compounds, as well as residues of compounds, that may be incorporated into elastomers and that may interact with effectors by means of forming a selective non-covalent or covalent bond, wherein the result of this is a change of volume of the elastomers.

The term effector is meant to comprise all compounds that are capable of interacting with the receptors while forming a selective non-covalent or covalent bond, wherein this leads to a change in volume of the elastomers.

In principle, it is possible to employ all compounds as effectors and receptors that are characterized in the literature with the term host-guest-molecule or supramolecular complex and that are capable of engaging in a host-guest-relation.

The term covalent bond is to be understood that an electron pair bond is formed between effector and receptor.

The term selective non-covalent bond thereby preferably is meant to be understood as bonds or interactions that are effected by means of ion pairs, by means of hydrogen bridge bonds, by means of dipole-dipole interactions, by means of charge-transfer interactions, by means of π-π- and C—H-π interactions, by means of cation-π interactions, by means of van der Walls interactions and dispersive interactions, by means of hydrophobic (lipophilic) interactions, by means of metal-complex formation, preferably with transition metal cations, as well as combinations of these types of this interactions.

It is particularly preferred in the sense of a selective non-covalent bond and the change in volume triggered thereby, if effectors and receptors interact in a complementary manner while simultaneously effecting several types of interactions. Complementary thereby means that the effectors and receptors are tuned with respect to each other in a manner so that they can effect a particularly strong non-covalent bond with each other. The more pronounced the complementarity between effectors and receptor is, i. e. the more they therefore fit together, the stronger is the non-covalent bond or, respectively, the more of the above mentioned interactions can be effected between effectors and receptors. In general, this manifests itself in an increase of stability of the system that forms out of elastomer and effector. This leads, in general, to a significant enhancement of the selectivity and of the response sensibility of the receptor modified elastomer.

Therein, it is also possible that several receptors are complementary to one effector, and/or one receptor is complementary to several effectors. For example, the carboxylic, the amine and the amide group as receptors can be complementary to a hydroxyl group contained in the effector.

It is also possible that a second effector is bound more strongly by means of the presence of a first effector. For example, the primary bond of metal cations can amplify the bond of secondary effectors, such as peptides, by means of interaction with the metal cations, wherein, simultaneously, interactions with other parts of the receptors bound to the polymer may be effected.

The term complementary groups also comprises that such groups may be replaced by groups that are structurally similar to the complementary groups or that are of the same structural family.

According to the present invention, the system, however, is preferably characterized in that the receptors of the receptor modified elastomers and the at least one effector are mutually complementary.

It is also possible that the non-covalent bond that has been initially formed between effector and receptor transforms into a covalent bond upon the approach of the at least one effector to the receptors of the at least one receptor modified elastomer that are complementary to at least one effector.

In a preferred embodiment, the formation of said covalent bond is effected with a velocity that is sufficiently high for the intended application.

In particular in the case of fast reactions, a covalent bond may also be formed without the possibility of a non-covalent bond having formed before.

One example for the formation of a covalent bond is the reaction of boric acid and sugars or carbohydrates, respectively, leading to the formation of boric acid esters. Therefore, systems are possible that comprise as effectors sugar or carbohydrate residues and that comprise as receptor modified elastomers such elastomers with boric acid residues as receptors.

Furthermore, it is possible according to the present invention, that selective non-covalent bonds as well as covalent bonds may be effected between effectors and receptors. For example, this is possible in case a mixture of different effectors is employed.

The object according to the present invention is also solved if not only one or several selective non-covalent or covalent bonds are formed between the at least one effector and the receptors, but if the receptors are also protonated or deprotonated. Therein, the effector can either also work as a protonation or as a deprotonation agent. However, it is also possible to add a protonation or a deprotonation agent, in addition, to the effector, for example via the medium used in the system.

In contrast to the elastomers known from the prior art that show an increase of the volume for one and the same elastomer either only under basic conditions or only under acidic conditions, the correspondingly selected receptor modified elastomers of the systems according to the invention show a change in volume using one and the same elastomer under basic conditions as well as under acidic conditions, wherein the volume change is an increase of volume. Generally, this sensitivity against changes in the pH-value is high, leading to an almost symmetric increase in volume above and below the physiological pH-value.

Therefore, the system according to the invention is also characterized in that the change in volume of one and the same elastomer in an acidic and in a basic medium consists of an increase in volume, respectively.

Upon bringing into contact the receptor modified elastomers with the effectors, the latter penetrate into the polymer skeleton of the elastomers, wherein the volumetric change of the elastomer is effected by means of forming selective non-covalent or covalent bonds between receptors and effectors. The change in volume in preferably reversible.

In case the effectors are removed from the elastomer by means of change of concentration and/or by means of dissolution and/or by means of competition with a further effector, a reversal of the change in volume occurs.

In case a covalent bond is formed between the at least one effector and receptors out of the initially formed selective non-covalent bond, this covalent bond is preferably also capable of being cleaved, if necessary by means of adding substances that are capable of cleaving the bond. For example, ester bonds may be re-cleaved by means of adding a base or a hydrolytically acting catalyst.

It is not necessary that the effectors are being used in a medium upon bringing them into contact with the receptor modified elastomers. This applies in particular in case they are already present in liquid or in gaseous form. In order to effect an even penetration of the effector molecules into the polymer skeleton, however, the use of a medium in which the effectors are present dissolved, suspended or dispersed, is of advantage. The medium may be a liquid and/or a gas. The medium than has the task to wet the elastomers and to start the swelling process, but not to dissolve them.

In this embodiment, the system is also characterized in that the at least one effector comprises, as a medium, a liquid or a gas or a liquid and a gas, the elastomer is not being dissolvable therein.

If a liquid is used as a medium, this may be polar as well as non-polar. As a preferred liquid, water is used since most applications of the system are based on the use in an aqueous environment. In this case, preferably, the elastomers are of hydrophilic nature, i. e. they comprise hydrophilic groups. If applicable, the receptors may also be of hydrophilic nature. For applications in non-polar media, the elastomers may be modified by means of introducing hydrophobic groups or lipophilic (co)polymers. In a preferred embodiment, said modification is effected by means of incorporating long chains of alkyl residues.

As receptors, the elastomers preferably comprise residues of compounds that are selected from the group comprising amines, polyamines, acids, crown ethers, cyrptands, spherands, polyalkylene gycolether, polyamides, lactames, imides, urea, guanidines, aromatic/heteroaromatic compounds, calixarenes, resorcinarenes, cyclophanes, paracyclophanes, rotaxanes, catenanes, polyrotaxanes, polycatenanes, cavitands, cycloveratrylenes, cyclodextrins, peptides, proteins, metal complexes or mixtures of two or more thereof. Furthermore, biogenic receptors or parts thereof may be used as well, including proteins and nucleid acids.

Effectors preferably comprise substances selected from the group comprising anorganic acids, carboxylic acids, amines, vicinal amines, polyamines, amino acids, peptides, nucleosides, nucleotides, nucleic acids, biogenic effectors, steroids, Lewis acids, Lewis bases; alkaline, earth alkaline and transition metal cations; anions, or mixtures of two or more thereof.

The receptors and effectors are tuned with respect to one another so that they are preferably complementary to each other. Some examples for such embodiments are shown in the following:

In one of these embodiments, the receptors of the receptor modified elastomers comprise amine and/or polyamine residue and the at least one effector comprises acids and/or anions.

In a further embodiment, the receptors of the receptor modified elastomers comprise vicinal di- and/or polyamine residues and the at least one effector comprises transition metal cations.

In a further embodiment, the receptors of the receptor modified elastomers comprise acid residues and the at least one effector comprises amines and/or cations.

In a further embodiment, the receptors of the receptor modified elastomers comprise crown ether- and/or cryptan- and/or spherand-residues and the at least one effector comprises alkaline metal and/or alkaline earth metal ions and/or amines and/or aminoacids and/or peptides.

In a further embodiment, the receptors of the receptor modified elastomers comprise polyethylene glycol ether- and/or lariate ether-residues and the at least one effector comprises alkaline metal and/or earth alkaline metal ions and/or amines and/or aminoacids and/or peptides.

In a further embodiment, the receptors of the receptor modified elastomers comprise polyamide and/or lactames and/or imides and/or urea and/or guanidines and the at least one effector comprises anions and/or amides and/or peptides and/or alkaline metal and/or earth alkaline metal ions.

In a further embodiment, the receptors of the receptor modified elastomers comprise aryl and/or hetero aryl residues and the at least one effector comprises aromatic compounds and/or nucleosides and/or nucleotides and/or aromatic amino acids and/or peptides.

In a further embodiment, the receptors of the receptor modified elastomers comprise calixarene and/or resorcin aren residues and the at least one effector comprises aromatic compounds and/or amines and/or acids and/or nucleotides and/or steroids and/or amino acids and/or peptides.

In a further embodiment, the receptors of the receptor modified elastomers comprise cyclophane and/or cycloveratrylene residues and the at least one effector comprises aromatic compounds and/or amines and/or acids and/or nucleotides and/or steroides and/or amino acids and/or peptides.

In a further embodiment, the receptors of the receptor modified elastomers comprise residues of cyclodextrine and/or alkyl groups and the at least one effector comprises aromatic compounds and/or amines and/or acids and/or nucleotides and/or steroids and/or amino acids and/or peptides.

In a further embodiment, the receptors of the receptor modified elastomers comprise aromatic peptide residues and the at least one effector comprises aromatic peptides and/or aromatic compounds.

In a further embodiment, the receptors of the receptor modified elastomers comprise metal complexes and/or cyclophane residues and the at least one effector comprises Lewis bases and/or anions.

In a further embodiment, the receptors of the receptor modified elastomers comprise polypeptide and/or protein residues and the at least one effector comprises biogenic effectors and/or inhibitors and/or nucleic acids.

In a further embodiment, the receptors of the receptor modified elastomers comprise cyclo peptide residues and the at least one effector comprises alkaline metal and/or alkaline earth metal ions and/or amines and/or biogenic effectors.

It is preferred that the receptors of the receptor modified elastomers contain nitrogen. Such receptors are preferably of amine and/or of amide nature.

The term biogenic effectors is meant to be understood to comprise all synthetic or natural compounds having a physiological effect on a living plant or animal organism. Preferably, these are amino acids, oligopeptides, proteins, nucleic acids and their components, glucoproteins, antigenes, antibodies, carbohydrates, enzymes, co-enzymes, hormones, alkaloids, steroids, metabolites, viruses, micro-organisms, ingredients of plant and animal tissue, ingredients of blood, plasma or serum, cell disruptions, lecithines, as well as synthetic active ingredients such as pharmaceutical agents and pesticides, or toxins or toxic compounds, respectively.

Synthetic active ingredients preferably comprise substances that have an effect on the nervous system (antipsychotic drugs, barbiturates, analeptics, analgetics, local and general anaesthetics, muscle relaxants, anti-convulsants, anti-Parkinson-agents, antimetics, substances attacking via gangliae, substances attacking via the sympathicus, substances attacking via the parasympathicus); substances affecting the hormonal system (hormones of hypothalamus, hypophysis, thyroid; parathyroid and renal hormones; thymus hormones, substances affecting the organpancreatic island, adrenal gland; substances affecting the gonads); substances affecting mediators (histamines, serotonine, eicosanoides, platelets-activating factors, quinines); substances affecting the cardiovascular system; substances affecting the respiratorial system (antiasthmatics, antitussives, expetorantial, surfactants); substances affecting the gastrointestinal system (digestions enzymes, hepatitica), substances affecting the renal system and the urinary passages (diuretica); substances affecting the eye (ophthalmologica); substances affecting the skin (dermatotherapeutics); substances for preventing and treating infectious diseases (pharmaceuticals with antibacterial effect, antimycotics, chemotherapeutics for viral and protzoic diseases, anthelminthics); substances affecting malign tumors (antimetabolites, zytostatics, topoisomerase-blocking agents, mitosis blocking agents, zytostatically acting antibiotics, hormones and hormone antagonists); substances affecting the immune system and immunologically active substances (serums, immunomodulators, immunosuppressiva).

As pesticides, herbicides, insecticides, pesticides and fungicides are mentioned in particular.

As exemplary compounds and classes of compounds the following are disclosed: phenothiazine and phenothiazine-analogue substances, butyrophenones and diphenylbutyl piperidines, benzamides, benzodiazepines, hydoxytryptophanes, coffeines, amphetamines, opiates and morphines, phetidines and methadones, salicyl- and acetylsalicyl acid derivatives, arylpropion acid derivatives, antrhanil acid derivatives, aniline derivatives, pyrazole derivatives, sulfapyridines, hydroxychloroquines and chloroquines, penicillamines, N-methylated barbiturates and thiobarbiturates, dipropyl acetic acids, hydantoines, dopamines, noradrenolines and adrenolines, ergot alkaloids, carbamine acid derivatives, phosphoric acid ester, belladonna alkaloids, hormones of the hypophthalmus, HVL-hormones, hormones of the posterior pituitary, thiouraciles and mercaptoimidazoles, sulfonylurea, histamines, triptanes, prostaglandines, dipyradimoles, hirudines and hirudine derivatives, thiazides, psoralenes, benzoylperoxides and azeleinic acid, vitamin A, vitamin K, vitamin B₁, B₂, B₆, nicotine acid amides, biotines, vitamin B₁₂, vitamin C, halogen compounds, aldehydes, alcohols, phenols, N-heterocyles, pyrethrines and pyrethroides, phosphoric acid ester, thiol phosphoric acid ester, carbamin acid ester, β-lactames, aminogylcosides, tetracyclines, fluorchinolones, oxazolidinones, diaminobenzyl pyrimidines, pyrazinamides, griseofulvines, aziridines, actinomycines, anthracyclines, zytocines, monoclonal and polyclonal antibodies.

The receptor modified elastomers of the system can react selectively with effectors so that even isomeric effector molecules, for example configurational isomers or stereo-isomeric effector molecules effect clearly distinguishable changes in volume. This behavior was not predictable and is therefore surprising.

For example, in the system according to the invention, phthalic acid, isophtalic acid and terephtalic acid as effectors may effect different changes in volume in one and the same elastomer.

The change in volume can be evoked by several effectors simultaneously. Therefore, it is possible, by combining different receptor groups in the elastomer, to obtain, for example, a macroscopically usable signal, for example for setting free an active ingredient as two different effectors exceed or fall below a predetermined concentration.

For example, it is possible to effect the change in volume of the elastomers by sulfate anions, wherein this is controlled by means of a second effector, such as sodium benzoate. It is not expected that this control can be limited to a sharply limited range of concentration.

As further systems that show this behavior, sulfate/adenosine monophosphate and phosphate/adenosine monophosphate can be quoted.

In the examples as quoted above, the formation of the non-covalent bond is mainly effected by means of ion-pairs and van der Waals interactions.

Further examples for such interactions are interactions between an elastomer that comprises as receptors residues of azamacrocycles, and effectors that consist of multiple charged anions. For example, anions of the isomer form of 1,3,5-trimethyl-2,4,6-cyclohexane tricarboxylic acid and anions of the isomeric benzene tricarboxylic acid in elastomers that contain as receptors protonated forms of the azamacrocycle [21]anN₇, may evoke a different change in volume.

Furthermore, the mono-, di- and triphosphate of nucleotides and different nucleic bases in elastomers that contain, as receptors, cyclophanes that contain ammonium groups, may evoke distinct changes in volume.

It is also conceivable that citrate anions in elastomers that contain, as receptors, protonated forms of 1,3,5-triethyl-2,4,6-tris(3,4-dihydro-5H-1-aminomethyl) benzene may trigger a specific change in volume.

As further examples, anions of trans-aconitic acid can be named, triggering a specific change in volume in elastomers that contain, as receptors, tetracationic pyridinium salt residues containing four pyridine units, in which one nitrogen atom of one pyridine ring is linked, respectively, with one carbon atom of one further pyridine ring.

It is also possible that nucleic bases in elastomers trigger a specific change in volume, wherein said nucleic bases contain as receptors protonated cyclodextrine residues that are substituted with aminomethyl-groups.

Cationic choline and choline acetate are able to trigger a specific change in volume in elastomers that contain, as receptors, anionic resorcinarene residues.

As has already been discussed above, the interaction between the receptor and effector may also be effected via hydrogen bridge bonds.

For example, ammonium compounds in elastomers that contain crown ether residues as receptors can trigger a specific change in volume.

As has already been discussed above, the interaction between receptor and effector can also be effected via a cation-π-electron-interaction.

For example, alkaline metal and organic ammonium cations in elastomers that contain, as receptors, cucurbiturile- crown ether-, krytand- or polyethylene glycolether-residues can trigger a specific change in volume.

Furthermore, ammonium cations in elastomers, containing calixarene residues as receptors can trigger a specific change in volume.

As has already been described above, the interaction between receptor and effector can also be effected via van der Waals-interaction.

For example, nucleotides in elastomers, containing azapyrenium residues as receptors can trigger a specific change in volume.

Furthermore, electron-rich compounds, such as phenolic derivatives, in elastomers containing tetra cationic cyclophane-residues as receptors, can trigger a specific change in volume.

Furthermore, fullerenes in elastomers containing calixarene residues as receptors, for example a sulfonated calix[8]arene-residue, can trigger a specific change in volume.

As has already been described above, the interaction between receptor and effector can also be affected via a hydrophobic (lipohilic) interaction.

For example, differently substituted 1,4-benenes in elastomers containing cyclophane residues as receptors, can trigger a specific change in volume.

Chirale receptor groups may be introduced into the elastomers, wherein the use of optically active effectors enables a change in volume that is induced enantio selectively. Upon introducing several centers of chirality, correspondingly, it is possible to induce a change in volume that is diastereo selectively induced.

It is also possible, to introduce groups into the elastomers that react to redox processes. The change in volume as triggered by the effector then depends on whether said group is present in oxidized or in reduced form. This behavior can be used for switching processes. One group that reacts to redox processes is, for example, the —S—S-group that may be transferred into the sulfide group. Thereby, changes in volume may be triggered by substances that act as oxidizing or as reducing agent, as well as by applying an electric voltage.

It is also possible, to manufacture receptor-modified elastomers that contain mostly and preferably only amine and/or amide receptors as receptors. For example, it is possible to incorporate receptors into elastomers that contain one, two, three or more nitrogen atoms. Such elastomers then preferably comprise long chain amine residues or (—NH—CH₂—CH₂—NH—)— or (—NH—CH₂—CH₂—NH—CH₂—CH₂—NH—)— units in the receptors. Such residues and units are, for example, contained in compounds such as dodecylamine, diethylene triamine, triethylene tetramine or tetraethylene pentamine that can be incorporated as receptors into elastomers. These elastomers also are capable of showing the above described increase in volume in systems that contain protons or hydroxy ions.

The receptor-modified elastomers of the system according to the invention are also capable to show significant changes in the optical domain upon as affected by the effectors upon the change in volume. Preferably, upon changing the volume, transparency, index of refraction, refraction of light and/or scattering of light of the elastomer may change simultaneously.

Therefore, the system is also characterized in that upon, bringing into contact the at least one effector with the receptor-modified elastomer in addition to the volume also the optical properties of the receptor-modified elastomers change.

Preferably, the receptors are evenly distributed over the polymer chains of the elastomer. By using such an arrangement, a volume change is achieved, that extends evenly over the entire elastomer. This is of advantage for the intended applications.

Preferably, the receptor-modified elastomers of the system are therefore prepared according to a suitable process, so that the receptors are evenly distributed within the entire elastomer.

During the manufacture of the elastomers, the receptors are preferably introduced in a polymer by means of reacting at least one suitable functionalized polymer with at least one receptor.

According to less preferred embodiment of the present invention, it is, however, also possible to first react at least one functionalized polymer with at least one receptor and to let the receptor-modified monomer obtained thereby react further to a polymer.

Thereby, the receptors are either introduced after the preparation of the polymer that forms the basis of the elastomer are introduced or already prior to the reaction to form the polymer.

Therefore, the system is also characterized in that it is manufactured according to a process comprising at least one of steps (i) or (j):
(i) reacting at least one functionalized polymer with at least one receptor, or
(j) reacting at least one functionalized monomer with at least one receptor and subsequently reacting the at least one receptor-modified monomer obtained thereby to arrive at a polymer.

In both cases, the initial products that form the basis for the steps have to contain functionalities that allow the covalent binding of the intended receptors either to a polymer or to at least one monomer.

As functionalities for coupling receptors to polymer compounds of step (i) or to monomers of step (j), preferably the following groups are suited:

—OH, —NRH, —NH₂, —COOH, —COOR, —CONH₂, —CONHR, —SH, —CN, —SCN, —NCS, —C₆H₄—CH₂X (X═OH, —NRH, —NH₂, Cl, Br), —OP(O)(OR)₂, —OSO₂ (OR), wherein R is preferably a hydrogen-, aryl-, heteroaryl- or alkyl residue.

Polymers according to the present invention are polymerisates, polycondensates as well as polyaddition compounds.

As examples for polymers of step (i) with functionalities for the introduction of receptors, the following may be employed preferably: polyacrylic acid alkylester, polymethacrylic acid alkylester, polyinvylalcohol, polyethyleneimine, polyalkylamine, polyvinylamine, polyviny limidazole, polyglucosamine (chitosane), copolymerisates of polymaleic acid anhydrid and α-olefines, peptides, modified proteins, polysaccharides and cellulose as well as mixtures of two or more of these polymers or copolymers. However, polymers of polyphenylene-, polyester-, polyamide-, polyether-, polyetherketon-, polyethersulfonic-, polyurethane- or polysiloxylsilane type can be employed as well as long as they are functionalized with the groups as described.

Therefore, the system is also characterized in that the functional groups of the polymer of step (i) or the functional groups of step (j) are selected from the group comprising one or more of the following groups: —OH, —NRH, —NH₂, —COOH, —COOR, —CONH₂, —CONHR, —SH, —CN, —SCN, —NCS, —C₆H₄X (X═OH, —NRH, —NH₂, Cl, Br), —OP(O)(OR)₂, —OSO₂ (OR), wherein R means a hydrogen-, aryl-, heteroaryl- or alkyl residue.

Preferably, the reaction according to step (i) is performed in a homogeneous, heterogeneous or microdisperse phase since this way of processing allows for an even introduction of the receptors into the polymer.

Suitable solvents preferably are aprotic solvents. Examples are dimethyl sulfoxide, dimethyl formamide, dimethyl acetamide, methyl-t-butylether, tetrahydrofurane or sulfolane. Nitromethane may also be used advantageously. Mixtures of solvents containing such solvents may also be used. In order to achieve a quick and even reaction, the educts are dissolved under heating, if necessary.

The reaction of step (i) may also be catalyzed. A suitable catalyst, preferably for reactions with functionalities such as the hydroxyl- or the amino group is, for example, dimethyl aminopyridine.

The receptors can be reacted with the functional groups of the polymer in step (i) either directly, in case they are sufficiently reactive. However, they can also be, if applicable, used in the form of derivatives that are capable to react with the functional groups.

For example, amines as receptors may be directly reacted with polymers that contain ester groups, wherein receptor modified elastomers with amide groups result.

Preferably, primary or secondary amines are reacted with the polymers containing ester groups, either alone, respectively, or also in a mixture.

It is particularly preferred to react primary amines with the polymers containing ester groups.

In a preferred embodiment, primary amines are such amines that are substituted with an aliphatic, cycloaliphatic or aromatic residue, or the mixtures thereof. Herein, the carbon chains or the carbon rings, respectively, may be interrupted or substituted by one or more of the groups —O—, —S—, —NR′—, —CO—, SO₂— or mixtures of two or more thereof, wherein R′ preferably means a hydrogen-, alkyl-, aryl- or heteroaryl residue.

As examples for such compounds the following are given: aliphatic amines, such as methyl- and ethylamine, prepyl-, butyl-, pentyl-, hexyl-, heptyl- and octylamine, dodecylamine, N,N-dimethyl aminopentylamine, N-aminoethyl pyrrolidon; polyamines such as ethylene diamine, diethylene triamine, triethylene tetramine and tetraethylene pentamine; aromatically substituted methylamines, such as benzyl, naphtylmethylamine, chinoline methylamine, pyridylmethylamine; aromatic amines, such as aniline, pyridylamine, chinolineamine, furfurylamine, indolamine, porphyrine. As a chinolineamine, primachine may be named, as indolamine seraotonine or tryptamine can be named.

Also further compounds, that are substituted with an amino group or an amino methyl group may be named, such as the correspondingly substituted crown ethers or spherands, azamacrocycles, cyclophanes, paracyclophanes, cyclodextrines, resoorchinearenes, calixarines.

Different receptors may be introduced by reacting the corresponding receptors or their derivatives, respectively, simultaneously or subsequently with the educt polymers in step (i). In an analogous manner thereto, monomers that comprise different receptors may be co-polymerized with each other in step (j).

Further processes for the manufactures of derivatized polymers are disclosed in WO 00/32649 and can be employed for the manufacture of the elastic swellable elastomers necessary for the present process. The processes described therein are particularly suited for the targeted introduction of up to three different receptor units into a polymer.

In step (i), preferably, polymerisates are used as polymers. Apart from copolymerisates, homopolymerisates may be used as well. Frequently, higher selectivities with respect to binding different effectors can be achieved by using homopolymerisates. Therein, the necessary flexibility of the polymerisates is preferably ensured by introducing polymerisate particles that do not carry receptors.

In an alternative to the process of step (i), the elastomers may also be manufactured in accordance with a process comprising step (j) by means of reacting at least one functionalized monomer with one receptor and subsequent reaction of the so-formed receptor-modified monomer to a polymer.

Therein, the monomer may be designed so that it is capable of polymerization, polycondensation or polyaddition.

Preferably, the functionalized monomer is olefinically unsaturated and the reaction of the receptor-modified monomer to arrive at the polymer is conducted by means of a polymerization. Preferably, therein the receptor-modified monomer is co-polymerized with at least one further monomer. Preferably, this at least one monomer also carries a receptor.

The receptor-modified monomers are thereby sensibly manufactured by means of reacting receptors or derivatives thereof with olefinically unsaturated compounds that are functionalized with the above-mentioned groups. Therein, the receptors are bound covalently to the monomer.

Preferably, (meth)acrylic acid esters are reacted with receptors to arrive at receptor-modified monomers. The monomers obtained thereby may then be polymerized or copolymerized, respectively.

As polymerization processes, the usual methods are applied, also, for example, a polymerization induced by radicals or ionic polymerization. It can be performed in solution or also as emulsion- or as suspension-polymerization. In case the polymerization is performed in solution, solvents are preferably used that dissolve the monomer or the monomers, but do not dissolve the polymerisate or the copolymerisate.

It is possible, for example to radically copolymerise a receptor-modified monomer with, for example, methylmethacrylate or acrylamide or N-vinyl pyrrolidone and/or the derivatives thereof, into which the desired receptors have already been introduced. Preferably, copolymerization is performed in a manner so that the velocities of homopolymerization and heteropolymerization are comparable.

Polymersation or copolymerization, respectively, may also be performed while adding network forming substances, such as divinylbenzene or di-, tri- or tetraacrylates, wherein the mechanical stability of the elastomer is increased. Suitable acrylates are, for example, bisphenol adiacrylate, pentaerythrite triacrylate, tetraethylene glykoldiacrylate or N,N′-methylene bisarcrylate.

A network, however, may also be achieved by means of using suitable α,ω-bifunctional compounds so long as these are suited for a reaction with the groups that are located on the polymer of step (i) or the monomer of step (ii). Preferably, polyamines can be reacted with polymers containing estergroups or carboxylic groups and with monomers. For example, diethyene triamine or triethylene tetramine may be used. Herein, the formation of networks is achieved by forming covalent bonds.

The formation of networks may either be achieved immediately upon introducing receptor groups into the polymer or the monomer that forms the basis for the elastomers, or by the subsequent reaction of the polymers with such compounds.

However, it is also possible to establish the formation of networks by means of non-covalent bonds. To achieve this, groups are introduced into the elastomer that may strongly interact with each other. For example, it is possible to achieve such an interaction via hydrogen bridge bonding. Suited are, for example, amide groups, that can interact with each other by means of complementary acceptor- and donor groups. By means of the effectors acting upon competitive groups in higher concentration, this network forming may be interrupted. Such effectors are, for example, protic effectors such as water or carboxylic acids. This effect also may be used for a change in dimension of the elastomers.

In case network forming reactions are performed, the degree of networking is preferably controlled so that it does not significantly limit the extensibility and therefore the potential changes in dimension of the polymers.

It is also possible, to bind the receptors to a polymer or a monomer by means of using acitivating agents. For example, polymers or monomers containing hydroxyl-, amine- or thiol groups may be transferred, preferably by using carboxylic acid anhydrides, into the corresponding carboxylic acid derivatives. These derivatives can then be reacted with receptors that work as nucleophiles. This results in receptor-modified polymers and monomers by way of cleaving off the carboxylic acid or the carboxylic acid amide or the thiol carboxylic acid, respectively.

The receptor-modified elastomers as obtained via steps (i) or (j) are preferably obtained with a solvent. Preferably, they are only slightly soluble therein or precipitate from said solvent upon cooling, respectively, since they form gels that are non-soluble or hardly soluble. If necessary, they may also be precipitated out of the solvent by means of adding further, non-soluble liquids. Afterwards they are isolated by means of filtration.

For the preparation of receptor-modified elastomers, it is also possible to add radiopaque substances in a known manner, such as tantalum, gold or platinum (WO 02/071994), or also lanthanoids. This can be of interest, for example, if the system of the invention is intended to be used to aid in radiographic examinations or in therapeutic applications.

It is also possible to add pore forming agents, such as salts, ice-crystals or sugar, in a known manner in the process of manufacturing receptor-modified elastomers (WO 02/071994). By way of forming pores, the access of the effector(s) to the receptors within the elastomer can be improved.

A further object according to the present invention is to also provide a process for the manufacture of the system that comprises as components at least one effector and one receptor-modified elastomer.

This process is characterized in that it comprises the bringing into contact of the at least one effector with the at least one receptor-modified elastomer.

In a preferred embodiment, the process is also characterized in that it comprises further more at least one of the steps (i) or (j):
(i) reacting at least one functionalized polymer with at least one receptor,
(j) reacting at least one functionalized monomer with at least one receptor and subsequently reacting the at least one receptor-modified monomer obtained thereby to arrive at a polymer.

With respect to the manufacture and the isolation of the receptor-modified elastomers it is possible, to immediately process them to be in shapes that are advantageous for the intended application. Preferably, they can be processed to result in thin films.

By means of the film thickness or the size of the polymer particles, it is preferably possible to control the response time of the receptor-modified elastomers with respect to the employed receptors. Generally, the response time will get shorter upon decreasing film thickness and decreasing size of the polymer particle; the response sensitivity will increase.

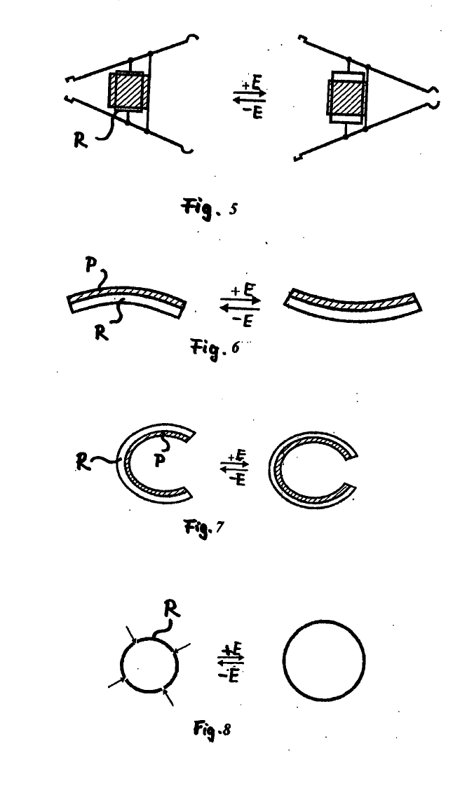

The elastomers can also be used in the form of composite polymers together with other polymers that are, preferably chemically inert, however, at any rate elastic. Thereby, increased mechanical stability as well as, in case of a suitable design, a greater macroscopic effect may be achieved. Suitable embodiments are shown by means of example in FIGS. 6 and 7.

Small elastomer parts are also of advantage for applications, for example in the shape of threads, microtubes or microspheres. By using these shapes, in general, faster and larger changes in volume can be achieved as compared to thicker films. Decreasing the size of the elastomer parts furthermore allows for a significant increase in sensitivity and therefore for a decrease in the concentration of the effectors necessary for the changes in volume. This is possible in particular if the bonding affinity between the receptors and the at least one effector is so large, that all receptors present in the elastomer part engage in a non-covalent bond with effectors. In this case, an almost linear relationship results between the size of the elastomer particles and the concentration of effectors necessary to achieve a certain change in volume. The degree of change in volume can be increased by means of increasing the number of receptors.

The elastomers are preferably suitable to be used for a multiple recognition of complex effector structures with several bonding sites for highly selective chemo-mechanical change in dimension. Herein, an increase in volume as well as decrease in volume as preferably evoked by means of the reversible dissociation of a selectively bound effector, may be used for applications.

The volume change as achieved by the effectors can be transferred into mechanical movements. Such movements can be the basis for switching processes as well as for actuators. A particular advantage of the novel receptor-modified elastomers can be seen in the fact that the change in volume as triggered by the effectors is sufficiently fast and also completely reversible for these applications. In particular, they do not need any further elements for the execution of switching signals and also no power supply. These properties are extraordinarily useful for the application.

An important novel property of the system according to the invention consists in the self control of macroscopic processes. Such processes are, for example, flow control, setting free of active ingredients or switching processes. For these processes, no additional sensors are necessary which would furthermore mostly require an external voltage supply. This is, because the elastomers themselves may be used advantageously as sensors.

It is also possible to use the change in transparency upon the change in volume, as mentioned above, for the purpose of sensoring.

Flow control is of interest for many potential applications. For example, it is of relevance in the area of medicine, for example for dialysis, for removing unwanted substances or in implants for dosing active ingredients. However, flow control is also important for technical processes such as separation of materials, wherein valves or pumps are used.

Flow control is possible, for example, with the devices as shown schematically in FIG. 1 to 4, wherein the receptor-modified elastomers have the functionality of valves.

In FIGS. 1 and 4, the receptor-modified elastomers R are processed to be tubings or tubes that may also contain an attachment in the shape of a funnel or an insert in the shape of a funnel.

Upon acting of the effector E, as a result, an increase of the diameter of the tubing upon the increase in volume is effected in the device of FIG. 1, wherein the flow of a substance that flows through the tubing is increased. Conversely, upon removing the effector, the elastomer contracts, thus reducing the flow through the tubing.

According to arrangement 4, the flow is controlled by means of a locking element V that is situated within the funnel. Upon action of the effector E on the funnel, said funnel expands thus increasing the flow. If the effector is taken away, the funnel contracts, thus reducing the flow through the locking element or interrupting said flow.

In arrangement 2, the receptor-modified elastomer R is situated within a tubing. Upon acting of the effector, it expands thus reducing or interrupting the flow within the tubing.

FIG. 3 shows an arrangement of a tubing with a funnel-shaped attachment containing a locking element consisting of the elastomer R. If an effector E acts upon the locking element, said locking element expands thus reducing the flow through the tubing, and vice versa.

The system according to the invention can also be used as an actuator.

In case of an arrangement as shown in FIG. 5, expansion of the elastomer R within a liquid medium under the effect of effector E leads to a mechanical movement that may be further transferred by means of levers.

The combination with a chemically inert polymer P, preferably a composite polymer, allows for an arrangement as is shown in FIGS. 6 and 7. Upon bringing into contact with an effector, only the elastomer R can expand, thus resulting in a reversible bending of the parts.

In particular with the devices according to FIGS. 6 and 7, it is possible to transfer mechanical changes obtained under the effect of effectors into signals, preferably into electromechanical, electrical and optical signals. Thereby, it is possible to measure chemically induced changes in length, for example of measuring strips in reactors or tubes, volumetrically and with high precision. Therefore, receptor-modified elastomers can also be used as sensors.

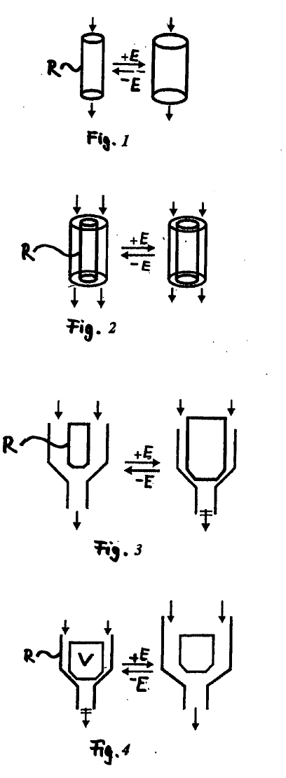

Also, the elements shown in FIGS. 1 and 2 can not only be used for active flow control, but also allow, if used as sensors, for the simultaneous measurement of substances that are contained within the flowing mass.

Furthermore, by using the novel elastomers, active ingredients may be set free out of suitable devices. Preferably, this is achieved by means of changes in the effector concentration in the environment of the elastomer, for example in bodily fluids. This effect may also be achieved by means of changes in the pH-value. Applications can be seen, for example, in therapy with respect to ulcer and tumor as well as in the field of radio-diagnostics. One potential further therapeutics application envisions the setting free of metal complex forming ligands, cytostatica or the dosage of insulin for controlling the level of glucose.

Conversely, uptake of materials, for example out of tissue, as induced by mechanical movements, is possible.

Apart from the schematically shown devices as shown in FIGS. 1 through 7, microspheres or microtubes are particularly suited for the setting free of active ingredients, wherein contraction or breach of thin parts sets free active ingredients out of pores. Therein, a contraction can also be achieved by means of replacement of a substance that is already present in the elastomer but bound in a weaker manner.

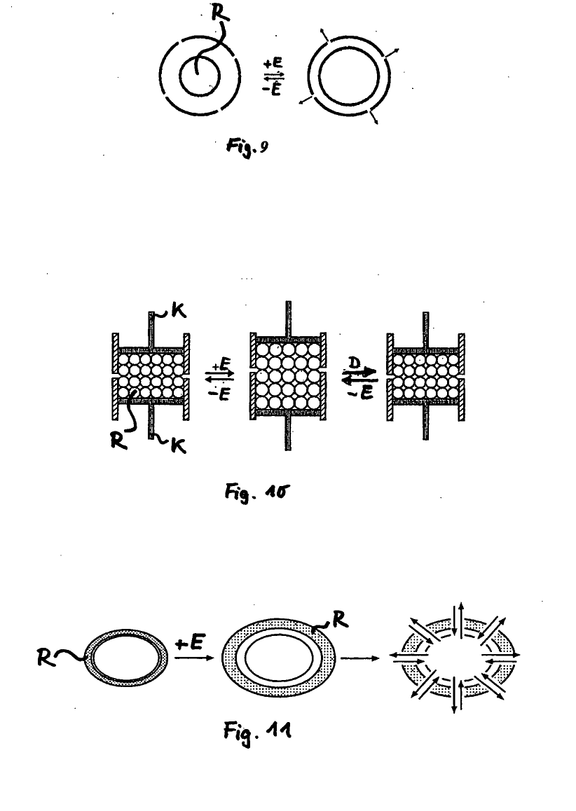

FIG. 8 shows an arrangement according to which the pores of a device are closed by means of an expansion of the elastomers R as effected by the effectors, thereby interrupting a setting free of active ingredients. On the other hand, in this case, it is also possible to interrupt the taking up of active ingredients by means of closing the pores by means of an expansion of the elastomers R as effected by the effectors.

Alternatively, by using a device that is built up with multiple shells, an expansion of cavity can also be used for setting-free. Herein, the active ingredient is replaced from a cavity the volume of which is reduced due to expansion. Such an arrangement is shown in FIG. 9.

The elastomers as described can also be used for the taking up or the removal, respectively, of unwanted substances from the environment, wherein said substances selectively work upon the receptors of the elastomers. For example, it is possible to remove toxic components out of tissue by using the arrangements as shown in FIGS. 8 and 9.

With the arrangement as shown in FIG. 10 it is possible to realize a setting free or absorption of substances as effected by a mechanical effect from the outside. Upon the effect of the effector on the elastomers R, said elastomers expand thus allowing to exert mechanical work via piston K, for example similar to a pumping function. Upon taking away the effector, the process can be arranged reversibly. Upon the effect of outer forces, such as compression or expansion, the process can be, in principle, reversed, while setting free or absorbing a substance.

Therein, ultrasound may also be employed to create inner pressure D.

It is also possible, to completely coat active ingredients with a film consisting of receptor-modified elastomers. Therein, polymer films maybe directly applied on to capsules, tablets, suppositories, etc. Upon bringing into contact with the effector or with several effectors, for example salts, the film may swell so strongly that the active ingredient can be dissolved or flushed out, for example by water, via the pores or fissures formed thereby. Such an arrangement is shown in FIG. 11.

A further possible application of the system according to the invention is the use as sealing material. In compliance with this application, the receptor-modified elastomers are applied into the opening that is to be sealed. Upon bringing into contact with an effector, the elastomers may swell so strongly as effected by a change in volume that the opening is tightly sealed. For example, the elastomers may be arranged in the shape of a sealing ring. Such an arrangement is shown in FIG. 12. Two tubes O1 and O2 that engage with each other are shown schematically in a longitudinal sectional view, wherein they are sealed against each other by means of a sealing ring of receptor-modified elastomers. Upon bringing into contact with the effector E the elastomer swells in such a strong manner that a sealing between the two tubes is effected.

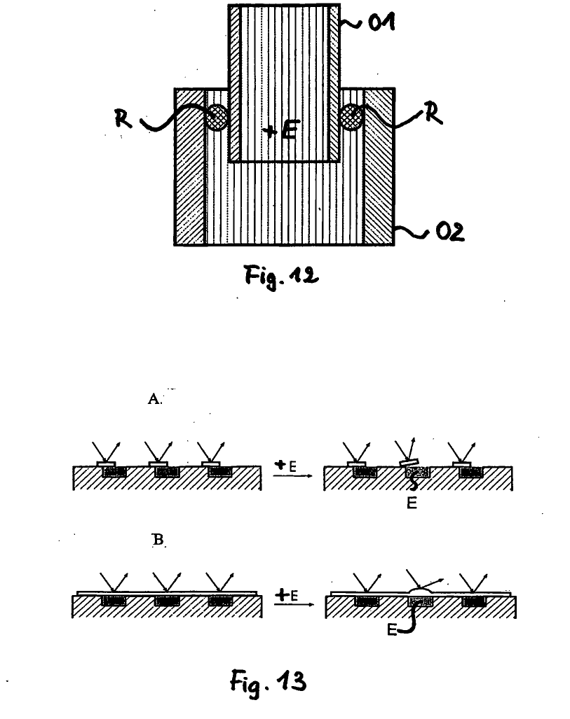

It is also possible to obtain so-called sensor arrays on the basis of receptor-modified elastomers, wherein these sensor arrays experience a change in volume upon being brought into contact with an effector. Herein, the mechanical movement that is triggered will be detected by means of deflection of light rays. Such an arrangement is shown in FIG. 13.

The effectors that in this case correspond to the substance that is to be analyzed are distributed in solution in individual cavities onto microtiter plates. The receptor-modified elastomer that contains a receptor for the searched-for effector is applied in arrangement A onto a tongue that is combined with an inert material, for example consisting of silicon or of plastic, wherein tongue and inert material experience a bending of the above described kind.

In an arrangement B that is alternative, the chemo-mechanical polymer is applied as a film over the entire cavities of the plate. The interaction with an effector E then leads, by means of change in volume of the film in that position, to a bulging with a resulting deflection of the light ray.

In a further embodiment, droplets with the effectors to be investigated are applied to a thin film of the chemo-mechanical polymer that is arranged on a flat plane. A change in volume that occurs selectively leads to a deflection of the light ray at that position.

In a further embodiment, smallest elastomer particles are employed that may be applied onto a flat plane out of the dissolved elastomer by means of removal of the solvent. A selective interaction with analytes it is brought in contact which leads, in this position, to a diverted deflection of the light ray.

By means of simultaneous irradiation with light that is guided in parallel, several thousand probes maybe examined on a small chip in a shortest time period, in case the deflected light is used for the generation of a two-dimensional pattern. Using this process, the marking with fluorescence dyes as necessary in common screening procedures, as well as the limitations connected therewith, are obsolete. Conversely, the process may also be used for the selection of effective receptor substances or combinations thereof out of the corresponding libraries. Correspondingly, novel “DNA” (gene)-chips may be made accessible by means of using chemo-mechanical polymers that are tagged with immobilized nucleotide sequences. Also, the potentially occurring change in optical properties of chemo-mechanical polymers, such as their transparency, can be used simultaneously for detection purposes. Arrangements as are shown schematically in arrangement A and B may also be manufactured by means of photolithographic methods as known from micro-systems techniques.

A further object according to the present invention is therefore the use of the system according to the invention for flow control, as actuator, as sensor, as sensor array, for setting free or taking up active ingredients, or as a sealing material.

In an advantageous embodiment, the effectors and receptor-modified elastomers of the system can be used for the manufacture of valves and pumps that are free of metals and are self-regulating, and are used, for example, for setting free or taking up active ingredients, as actuators, or sensors, or as a sensor array, or as sealing materials on the micro- and the nano-scale.

Furthermore, the present invention also relates to elements for flow control, for example a valve, or an actuator for the execution of mechanical movements, or to a sensor for chemical, mechanical, electrical, electromechanical, magnetic and optical signals, or to a sensor array, or to a device for setting free or taking up active ingredients, or to a sealing, that can be manufactured by using the system according to the present invention.

The aforementioned properties therefore render the novel system extraordinarily interesting for application potentials.

The invention shall now be further illustrated by means of examples.

### EXAMPLES

### Example 1

1 molar equivalent of poly(methyl)acrylic acid methylester was added to dimethyl sulfoxide (DMSO) in a flask with a reflux condenser. In short intervals, 1 molar equivalent of dodecylamine and 10 molar equivalents of diethylenetriamine were added under slow heating to 175° C. in an oil bath. After two hours, the mixture was cooled and three parts of water were added to the resulting mixture. The gel-type mass thereby obtained was washed five times with an excess of water, was dried on filter paper and subsequently dissolved in hot DMSO. The viscous solution was poured into glass-ware so that a least a 1 mm thick layer did result. The glass-ware was put into a vacuum dry oven at 90° C. over night. Subsequently, residue amounts of di-ethylenetriamine, water, and DMSO removed at a higher vacuum. The remaining films being as bright as water were peeled from the bottom of the glass-ware and were washed for several hours in multiple-deionized water. The material was contained in water and was used in the swollen state for changes in dimension by means of adding external effectors.

Pieces of the desired size were cut out of the swollen material. These pieces still being in the swollen state were dipped into solutions containing the effectors of interest after the adhering surface water has be removed. For films of a thickness of about 0.5 mm the half-life of the volume change mostly was two to four minutes. Therein, the half-life is the time period in which one half of the maximum possible change in volume occurs.

### Example 2

The elastomer as produced according to example 1 was exposed to different pH-values. The resulting changes in volume are summarized in the table. As a reference value, the volume change at a pH-value of 7 is used, since no change was detectable at this value.

| pH-value | increase in volume in % |
|---|---|
| 1 | 70 |
| 3 | 55 |
| 5 | 12 |
| 7 | 0 |
| 9 | 22 |
| 11 | 65 |
| 13 | 70 |

### Example 3

In this example, the volume change upon the acting of different effectors at a pH-value of 7 has been examined. The results are summarized in the following table.

| effector | change in volume in % |
|---|---|
| benzoic acid | <5 |
| phtalic acid | 30 |
| isophtalic acid | 40 |
| terephthalic acid | 61 |
| β-naphthaline carboxylic acid | >5 |
| phosphoric acid | >5 |
| uridine monophosporic acid | 13 |
| adenosine monophosphoric acid | 21 |

### Example 4

The experiment from example 3 has been repeated by simultaneously adding sodium hydroxide in the presence of the respective receptors.

| effector | change in volume in % |
|---|---|
| benzoic acid | 150 |
| phtalic acid | 59 |
| isophtalic acid | 110 |
| terephthalic acid | 150 |
| β-naphthaline carboxylic acid | 170 |
| phosphoric acid | 75 |
| uridine monophosporic acid | 126 |
| adenosine monophosphoric acid | 87 |

### Example 5

The elastomer produced according to example 1 has been treated with sodium sulfate as effector, wherein the changes in volume as a function of concentration as shown in the following table have been found.

| effector concentration | change in volume in % |
|---|---|
| 0 | 0 |
| 0.1 | 65 |
| 0.2 | 110 |
| 0.4 | 12 |
| 0.8 | 18 |
| 0.16 | 25 |
| 0.32 | 25 |
| 0.5 | 25 |

The experiment has been repeated, wherein 0.1 moles of sodium benzoate, respectively, has been added to the sodium sulfate solution.

| effector concentration | change in volume in % |
|---|---|
| 0 | 0 |
| 0.1 | 45 |
| 0.2 | 60 |
| 0.4 | 12 |
| 0.8 | 20 |
| 0.16 | 22 |
| 0.32 | 20 |
| 0.5 | 20 |

It is clearly discernible that the effect of the second effector (sodium benzoate) on the first effector (sodium sulfate) remains limited to a narrow range of concentration from approximately 0.1 moles/l to 0.3 moles/l.

### REFERENCE LIST FOR FIGS. 1 THROUGH 13

R receptor-modified elastomer
E effector
V locking element
P polymers or composite polymers
D pressure
K piston
O1, O2 tubes

FIGS. 1 through 4, respectively, schematically show a valve for flow control.

FIGS. 5 through 7, respectively, schematically show an actuator.

FIGS. 8 through 10, respectively, show the schematics of a device for setting free or taking up active ingredients, respectively.

FIG. 11 schematically shows an arrangement for setting free an active ingredient.

FIG. 12 schematically shows a sealing.

FIG. 13 schematically shows a sensor array.

1. System comprising at least one effector and at least one receptor modified elastomer, characterized in that a change in volume is effected upon bringing into contact the at least one effector with the at least one receptor modified elastomer by means of forming a selective, non-covalent or covalent bond between the receptors and the at least one effector of the receptor-modified elastomer.

2. System according to claim 1, characterized in that the change in volume is reversible or, also, non-reversible.

3. System according to claim 1 or claim 2, characterized in that the receptors of the at least one receptor-modified elastomer and the at least one effector are complementary with respect to each other.

4. System according to any one of the preceding claims, characterized in that the volume change of one and the same elastomer in an acidic and in a alkaline medium is a volume enlargement, respectively.

5. System according to any one of the preceding claims, characterized in that the at least one effector comprises a liquid or a gas or a liquid and a gas as a medium, in which the elastomer is non-soluble.

6. System according to any one of the preceding claims, characterized in that the effector comprises compounds that are selected from the group comprising anorganic acids, carboxylic acids, amines, vicinal amines, polyamines, amino acids, peptides, nucleosides, nucleotides, nucleic acids, biogenic effectors, steroids, Lewis acids, Lewis bases; alkaline, earth alkaline and transition metal cations; anions, or mixtures of two or more thereof.

7. System according to any one of the preceding claims, characterized in that the elastomers comprise as receptors residues of compounds that are selected from the group comprising amines, polyamines, acids, crown ethers, cyrptands, spherands, polyalkylenegycolether, polyamides, lactames, imides, urea, guanidines, aromatic/heteroaromatic compounds, calixarenes, resorcinarenes, cyclophanes, paracyclophanes, rotaxanes, catenanes, polyrotaxanes, polycatenanes, cavitands, cycloveratrylenes, cyclodextrins, peptides, proteins, metal complexes or mixtures of two or more thereof, as well as biogenic receptors or parts thereof, including proteins and nucleic acids.

8. System according to any one of the preceding claims, characterized in that, upon bringing into contact with the at least one effector, in addition to the volume, the optical properties of the receptor modified elastomer change as well.

9. System according to any one of the preceding claims, characterized in that it is manufactured according to a process comprising at least one of steps (i) or (j):
(i) reacting at least one functionalized polymer with at least one receptor,
(j) reacting at least one functionalized monomer with at least one receptor and subsequently reacting the at least one receptor-modified monomer obtained thereby to arrive at a polymer.

10. System according to claim 9, characterized in that the functional polymers or monomers contain (functional) groups selected from the group comprising one or more of the (functional) groups —OH, —NRH, —NH₂, —COOH, —COOR, —CONH₂, —CONHR, —SH, —CN, —SCN, —NCS, —C₆H₄—CH₂X (X═OH, —NRH, —NH₂, Cl, Br), —OP(O)(OR)₂, —OSO₂ (OR), wherein R means a hydrogen-, aryl-, heteroaryl- or alkyl-residue.

11. System according to claim 10, characterized in that the reaction of step (i) is performed in homogenous, heterogeneous or a microdisperse phase.

12. System according to claim 11, characterized in that the reaction is performed in a solvent selected from the group comprising dimetyl sulfoxide, dimethyl formamide, dimethyl acetamide, nitromethane, tetrahydrofurane, methyl-t-butylether, sulfolan, or mixtures of two or more thereof.

13. Process for manufacturing a system as defined in any one of the preceding claims, characterized in that the process comprises the bringing into contact of the at least one effector with the receptor-modified elastomer.

14. Use of a system according to any one of the claims 1 to 12 or use of a system manufactured according to claim 13 for flow control, as an actuator, as a sensor or as a sensor array, for setting free or taking up active ingredients, as a sealing material.

15. Element for flow control or as a actuator for performing mechanical movements or as a sensor for chemical, mechanical, electrical, electromechanical, magnetic or optical signals or as a sensor array or device for setting free active ingredients or taking up active ingredients, or for sealing, comprising a system according to any one of claims 1 to 12 or a system manufactured according to claim 13.
